(19) United States
(12) Patent Application Publication
(10) Pub. No.: US 2008/0182904 A1
(43) Pub. Date: Jul. 31, 2008

(54) Baclofen for Relapse Protection in Addiction

(76) Inventor: Olivier Ameisen, Paris (FR)

Correspondence Address: WILSON IP LAW, 5204 DELAFIELD AVE., BRONX, NY, 10471, US

(21) Appl. No.: 12/014,746

(22) Filed: Jan. 15, 2008

Related U.S. Application Data
(60) Provisional application No. 60/884,989, filed on Jan. 15, 2007.

(51) Int. Cl. A61K 31/195; A61P 25/32; A61P 25/34; A61P 25/36; A61P 25/24

(52) U.S. Cl. 514/567

(57) ABSTRACT

The present invention is directed a method to prevent relapse in a patient being treated for substance or behavioral addiction using baclofen. Baclofen can also be used to treat depression or other psychological conditions.

This application claims priority under 35 U.S.C. §119(e)(5) to U.S. Provisional Application No. 60/884,989, filed Jan. 15, 2007, which is incorporated herein in its entirety.

### FIELD OF THE INVENTION

The present invention is directed a method to prevent relapse in a patient being treated for substance or behavioral addiction using baclofen. Baclofen can also be used to treat depression or other psychological conditions.

### BACKGROUND OF THE INVENTION

A comprehensive treatment for alcohol-dependence takes advantage of the dose-dependent suppressive effects of baclofen in combination with complete suppression of craving and symptoms by challenge with the strongest cue—alcohol in the form of standard drinks—to obtain relapse-proof treatment, and to establish the relapse-interruption dose to stop relapse, should it take place, in a patient.

In 2004, Ameisen (Ref. 1) proposed a new model of treatment for alcohol-dependence (AD) based on a postulate that the dose-dependent, motivation-suppressing properties of baclofen for alcohol consumption in the animal could be transposed to humans and thereby rapidly and completely suppress craving and symptoms of AD effortlessly.

In contrast with current treatments, which aim to reduce craving and symptoms using various anti-craving agents, treatment with a gradual dose-escalation of oral baclofen lead to a complete indifference to alcohol's strongest cues (except the for the strongest cue, actually drinking alcohol because of the fear of relapse, which would have been undesirable to test). In Ameisen's study, the patient needed 270 mg/day of baclofen for craving to completely and permanently disappear.

The dose at which symptoms of alcohol dependence, including cravings for alcohol, are suppressed is called the symptom-suppressing dose (SSD) or craving-suppressing-dose (CSD). At SSD, Ameisen found that coexistent (comorbid preexisting) anxiety was concomitantly also greatly attenuated. The only side effect was somnolence and that was reduced by progressively reducing the baclofen dosage to 120 mg/day, a dosage which was sufficient for craving not to reappear over the complete nine months of the published study. That study proposed that the SSD in other patients be reached empirically, the criteria for correct CSD being the patient's feedback to the physician (Ref. 1).

This method of complete and prolonged pharmacological suppression of symptoms and consequences of AD using the unique dose-dependent suppressive properties of baclofen (Ref. 2) has been successfully reproduced in seven individual patients (Pascal Gache, University Hospitals of Geneva) and in one hundred patients (Otto Lesch, Medical University of Vienna).

However, none of the foregoing studies addressed alcohol relapse, by far the most frequent and the most severe complication of AD. Up to 90% of the patients who succeed in becoming abstinent relapse within the four years that follow the onset of abstinence. Moreover, no existing treatment, including the FDA-approved naltrexone and acamprosate, or the not yet approved medications--baclofen, topiramate or rimonabant, has shown any efficacy in preventing this potentially disastrous complication. For those patients who succeed in becoming abstinent, with or without medications, exposure to the strongest cue—the drink of alcohol—is not only not proposed to test whether a given medication is protective against relapse, but is strictly avoided because such patients are known to be highly vulnerable to relapse if they drink alcohol even on such medications.

At the time of the invention, the inventor was completely craving and symptom-free as a result of pharmacological intervention for AD. However, the risk of relapse while on baclofen remained unknown upon exposure to the strongest cue, namely alcohol consumption. Clearly, baclofen treatment conveys a certain degree of protection (e.g., via indifference to alcohol)—since strong visual and olfactory cues (sight and odors of drinks, bottles and bars, etc.) do not trigger reappearance of craving whatsoever.

Nevertheless, the strongest cue—the drink of alcohol (actually consuming alcohol)—is documented to reactivate craving when patients are treated with any of the other anti-craving medications. In fact, among the patients treated in Switzerland with baclofen, Dr. Gache reported that some patients, even at high-dose baclofen, did relapse. This suggested that a given dose of baclofen in a given patient is not necessarily protective.

It has now been surprisingly discovered that by empirically determining a patient's SSD or CSD of baclofen, that patient Accordingly, an experimental test was devised to determine the susceptibility of a patient to relapse while being treated for alcohol dependence using baclofen. The inventor (a physician and referred to herein as the patient), after more than one year symptom-free, decided to test whether relapse was possible on a baclofen regimen, at the baclofen dose that completely suppresses craving and symptoms. Moreover, it was recognized that should cravings re-occur, an optional dose of 40 mg of baclofen could be administered at once (Ref. 1).

### SUMMARY OF THE INVENTION

The present invention relates to preventing relapse of substance and/or behavioral addiction. In one aspect, the invention provides a method to prevent relapse in a patient being treated for substance or behavioral addiction by treating the patient with one or more escalating doses of baclofen for a time sufficient to reach a symptom-suppressing dose (SSD) of baclofen. The patient is then maintained at the SSD for a period of time until the patient is able to sustain indifference to the addicting substance or behavior. Once this state of indifference is obtained, the patient is assessed to determine whether cravings occur when that patient is challenged with a strong cue that stimulates one or more cravings associated with the addiction for which the patient is being treated. If cravings do not occur, then the patient is protected from relapse. If cravings continue, the baclofen dose is escalated as before until the patient is indifferent to the addicting substance or behavior and the challenge repeated until craving stops. Once cravings cease, the patient is maintained on the SSD which stops cravings and is thereby protected from relapse of the addiction.

In another aspect, the invention is directed to a method of protecting a patient against relapse which comprises treating the patient with baclofen to determine the SSD of baclofen for that patient, reducing the baclofen dose to determine the maintenance dose of baclofen for that patient, performing a test for relapse by challenging the patient with a strong cue for an addictive substance or addictive behavior, and monitoring cravings for several days until no cravings occur during a several day period following the challenge. The patient is then protected from relapse.

In accordance with the invention, these methods can be used for any kind of addiction, including but not limited to, addiction to alcohol, nicotine, cocaine, heroin or other opioid drug, any other addicting drug and for such behaviors that have an addictive component such as gambling, sex, bulimia, binge-eating disorders or obsessive-compulsive disorders. In a preferred embodiment, the present methods are used to treat alcohol dependence.

In a yet further embodiment, the invention provides a method to treat or ameliorate depression or other psychological condition which comprises administering baclofen to a patient in need of treatment for a time and in an amount to relieve the associated symptoms of the depression or the condition and to provide therapeutic benefit to the patient. The conditions that can be treated include symptoms of menopause, obsessive-compulsive disorder, ADHD, ADD, tensions headaches, anxiety and the like.

### DETAILED DESCRIPTION OF THE INVENTION

In accordance with the invention there is provided a method to determine that dose of baclofen which prevents relapse in a patient being treated for addiction, including but not limited to addiction to alcohol, nicotine, cocaine, heroine or other opioids, other addictive drugs, gambling, sex bulimia, binge-eating disorders, obsessive-compulsive disorders and the like. For example, initial baclofen therapy is instituted to suppress all manifestations of the disease and therapy can be done with dose escalation until the SSD for that patient is reached and the patient can effortlessly sustain an indifference to the addicting substance for some period of time (which can range from several days, to many weeks or many months). The patient's vulnerability to relapse is then tested after a stable period of at least several weeks being completely craving/symptom-free with non-reactivity to strong cues (visual, olfactory and the like).

The state of sustained indifference to the addicting substance or behavior by the patient can last or be maintained for several days, several weeks, or several months. Those of skill in the art can determine the appropriate time interval for achieving and maintaining indifference by assessing the cravings of the patient and the symptoms of the patient. This assessment is done without a challenge, and when the patient is indifferent to the addicting substance or behavior without effort, then the patient is ready for the challenge.

For alcoholism, vulnerability is tested with an alcohol-challenge test of about three to five standard drinks. The number of drinks can and should be varied (typically lower) if any adverse signs or cravings appear in the patient. If craving does not occur at all, the patient is protected at the SSD against relapse. If craving occurs, the patient is not protected against relapse and the SSD dosage needs to be increased and the test performed again until craving stops occurring altogether upon renewed alcohol-challenge (s). Once that state is achieved, the patient is protected against relapse.

For other addictions, the challenge must represent a strong cue for relapse to that particular addiction and must not present a health or other risk. For nicotine addiction, a strong cue can be smoking a cigarette or in a setting where the patient would have always smoked. For behavioral addictions, the strong cue can be a setting in which the patient would have normally acted out the addicting behavior. For example, if entering a bar always lead to smoking, then that might be a sufficiently strong cue to test for relapse. For gambling, one might have the patient participate in simulated gambling. For opiod or other addiction, one prefers not to test the patient by administering the addicting drug. In these cases, the social cues that usually accompanied the drug use can serve as a strong cue. Alternatively, offering the drug (without actually giving it) to patient, might be strong cue. While not essential, especially depending on the addiction, the patient is preferably under the care, supervision and/or observation of a physician or other health care worker at the time of challenge.

Such challenges, including the alcohol challenge, should be done under supervision of a physician.

Baclofen doses are determined in accordance with the invention, i.e., in a dose-dependent manner to that establishes the SSD or CSD for each patient. Baclofen can be used at from about 20 mg/day to about 500 mg/day, and preferably from about 100 to about 300 mg/day.

As another example, for a patient whose symptoms and craving had been completely suppressed with baclofen for a stable period of time, the use of a baclofen dose that is 50% higher than the long-term dose is able to help stop relapse.

In another embodiment of the invention, the physician treats a patient with baclofen to determine the SSD of baclofen for that patient, then reduces the baclofen to determine the maintenance dose of baclofen for that patient and then performs the test for relapse as described above and monitors cravings for several days. If no cravings occur during a several day period following monitored challenge, then the patient is protected against relapse.

A maintenance dose is the lowest dose of baclofen that suppresses cravings.

Suppression of symptoms can be assessed by self reporting to the physician and include but are not limited to the patient reporting that he/she has no cravings, no thoughts about the addicting substance, no preoccupation with the addicting substance, no dreams about the addicting substance and the like.

Baclofen can also be used as an anti-depressant with immediate action (unlike other known anti-depressants that may need a few weeks to show therapeutic efficacy it can also be used to treat symptoms of menopause, obsessive-compulsive disorder (OCD), attention-deficit, hyperactivity disorder (ADHD), attention deficit disorder (ADD) and tensions headaches. In the foregoing treatments, baclofen is administered for a time and in an amount to relieve the associated symptoms of the condition and to provide therapeutic benefit to the patient. Those of skill in the art can determine the dosage for baclofen, which can range from about 20 mg/day to about 500 mg/day, and from 100 mg/day to 300 mg/day.

It will be appreciated by those skilled in the art that various omissions, additions and modifications may be made to the invention described above without departing from the scope of the invention, and all such modifications and changes are intended to fall within the scope of the invention, as defined by the appended claims. All references patents, patent applications or other documents cited are herein incorporated by reference in their entirety.

### EXAMPLES

The susceptibility of a patient to relapse while being treated for alcohol dependence using baclofen was determined. The patient (himself a physician) had been symptom-free for more than one year and was on a baclofen dose of 120 mg/day (40 mg, three times per day). This baclofen dose completely suppressed craving and symptoms in the patient.

First challenge: During a social gathering, the patient had 3 standard drinks of alcohol (gin and tonic) over a few hours and evaluated the effects in the presence of a physician witness. The first drink was consumed slowly by sipping over some 40 minutes, without the slightest urge to drink it rapidly (and which had previously been the only way the patient would drink, e.g., typically consuming the drink within 5 minutes). A second drink was ordered, consumed and started to produce a pleasant mild euphoria. A third drink was begun but the patient was not able to finish it, something that would have been impossible during untreated alcohol dependence. At the time, the patient felt no desire to finish the third drink, felt a bit high and maintained the ability to chat. After a night's sleep, the patient woke up without any feelings of remorse or fear. Most strikingly, the patient had no desire or thought to drink whatsoever. The following hours were unremarkable. No alcohol thoughts or dreams occurred in the following weeks.

Second challenge: The patient repeated the experiment approximately one month later with an even stronger challenge: five drinks. Again in the presence of another physician, the patient succeeded in having 5 drinks of vodka and tonic over 6 hours. The results were identical until the next day in the afternoon when a bout of craving was experienced. Administration of 40 mg of baclofen suppressed the craving within the hour. The craving reappeared again several hours later, suggesting that the alcohol had reactivated the craving cycle and requiring a higher level of baclofen-blockade. Accordingly the daily dosage of baclofen was increased from 40 mg three times daily, to 60 mg three times daily (180 mg/day). On that regimen, craving was again completely suppressed. After six days, the dosage was progressively reduced to 120 mg/day and cravings did not reoccur. Accordingly, symptom suppression is dose-dependent and may require higher dosage when challenge is important.

Third challenge: To test if a higher than routine dose of baclofen can prevent cravings, even in the presence of a massive amount of alcohol such as is ingested during heavy drinking or during active relapse, the patient's baclofen dose was reduced 24 hours prior to consuming an alcoholic beverage. The patient took 30 mg baclofen in the morning, a second such dose 8 hours later and a few hours later (in the evening) took dose of 80 mg baclofen at the same time as beginning to drink large amounts of alcohol as could reasonably tolerated, expecting that to be about a fifth of Scotch. The alcohol was consumed over several hours and the next day, actual consumption was measured to be four fifths of the bottle. Surprisingly, despite a mild hangover, the patient experienced no craving and no desire whatsoever to drink liquor. The patient continued treatment with 60 mg baclofen three times a day (180 mg/day) for six days, returned to the maintenance dose of 120 mg/day and has not experienced any craving for more than one year. The patient is able to have an occasional drink in a non-dependent fashion.

### REFERENCES

1. Ameisen O. Complete and prolonged suppression of symptoms and consequences of alcohol-dependence using high-dose baclofen: a self-case report of a physician. Alcohol Alcohol. 2005 March-April;40(2):147-50. Epub 2004 Dec 13.

2. Ameisen O. Naltrexone treatment for alcohol dependency. JAMA. 2005 August 24;294(8):899-900; author reply 900.

I claim:

1. A method to prevent relapse in a patient being treated for substance or behavioral addiction which comprises
(a) treating said patient with one or more escalating doses of baclofen for a time sufficient to reach a symptom-suppressing dose (SSD) of baclofen for said addiction;
(b) maintaining said patient at said SSD for a period in which said patient can sustain indifference to said addicting substance or behavior;
(c) assessing whether cravings occur upon challenging said patient with a cue that stimulates one or more cravings associated with said addiction.
(d) if cravings continue, repeating steps (a)-(c) until craving stops upon challenge;
(e) and maintaining said patient on that SSD which stops cravings and thereby prevent relapse of said addiction.

2. The method of claim 1, wherein said addiction is to alcohol, nicotine, cocaine, heroin, another drug, gambling, sex, bulimia, binge-eating disorders or an obsessive-compulsive disorder.

3. The method of claim 1, wherein said patient can sustain indifference to said addicting substance or behavior for several days, several weeks, or several months.

4. The method of claim 1, wherein said cue is the addicting substance or the addicting behavior.

5. The method of claim 1, wherein said addiction is alcohol-dependence.

6. A method to protect a patient against relapse which comprises treating a patient with baclofen to determine the SSD of baclofen for that patient, reducing the baclofen dose to determine the maintenance dose of baclofen for that patient, performing a test for relapse by challenging the patient with a strong cue for an addictive substance or addictive behavior, and monitoring cravings for several days until no cravings occur during a several day period following the challenge.

7. The method of claim 6, wherein said addiction is to alcohol, nicotine, cocaine, heroin, another drug, gambling, sex, bulimia, binge-eating disorders or an obsessive-compulsive disorder.

8. The method of claim 6, wherein said addiction is alcohol-dependence.

9. A method to treat or ameliorate depression or other psychological condition which comprises administering baclofen to a patient in need of treatment for a time and in an amount to relieve the associated symptoms of the depression or the condition and to provide therapeutic benefit to the patient.

10. The method of claim 9, wherein said conditions are menopause, obsessive-compulsive disorder, ADHD, ADD and tensions headaches.
